(19) United States
(12) Patent Application Publication
(10) Pub. No.: US 2013/0025000 A1
(43) Pub. Date: Jan. 24, 2013

(54) PROMOTER FOR REGULATION OF GENE EXPRESSION IN PLANT ROOTS

(71) Applicants: Tong Zhu, Durham, NC (US); Vance Cary Kramer, Durham, NC (US); Anthony Todd Richmond, San diego, CA (US)

(72) Inventors: Tong Zhu, Durham, NC (US); Vance Cary Kramer, Durham, NC (US); Anthony Todd Richmond, San diego, CA (US)

(21) Appl. No.: 13/514,119

(22) PCT Filed: Dec. 7, 2010
(86) PCT No.: PCT/US10/59166

§ 371 Date: Jun. 26, 2012

Related U.S. Application Data
(60) Provisional application No. 61/287,230, filed on Dec. 17, 2009.

(51) Int. Cl. C12N 15/113; A01H 5/10; A01H 5/00; C12N 15/82; C12N 5/10

(52) U.S. Cl. 800/279; 536/24.1; 435/320.1; 435/419; 800/298; 800/320; 800/320.3; 800/322; 800/317.4; 800/314; 800/320.2; 800/312; 800/317.3; 800/306; 800/320.1; 800/287

(57) ABSTRACT

The invention is directed to a promoter isolated from maize. The promoter of the invention have particular utility in driving root preferred expression, specifically root-cap expression, of heterologous genes that impart increased agronomic, horticultural and/or pesticidal characteristics to a given transgenic plant. The invention is also drawn to DNA molecules comprising the promoter of the invention and transformed plant tissues containing DNA molecules comprising a promoter of the invention operably linked to a heterologous gene or genes, and seeds thereof.

### FIELD OF THE INVENTION

The invention relates generally to the field of plant molecular biology and the regulation of gene expression in plants. The invention discloses nucleic acid sequences from Zea mays (corn) containing a regulatory element, such as a promoter. More specifically, the invention relates to the regulation of gene expression in plant roots with specificity to the root cap.

### BACKGROUND OF THE INVENTION

Manipulation of crop plants to alter and/or improve phenotypic characteristics (such as productivity or quality) requires the expression of heterologous genes in plant tissues. Such genetic manipulation has become possible by virtue of two discoveries: the ability to transform heterologous genetic material into a plant cell and by the existence of promoters that are able to drive the expression of the heterologous genetic material.

It is advantageous to have the choice of a variety of different promoters so as to give the desired effect(s) in the transgenic plant. Suitable promoters may be selected for a particular gene, construct, cell, tissue, plant or environment. Promoters that are useful for plant transgene expression include those that are inducible, viral, synthetic, constitutive (Odell et al., 1985, Nature 313: 810-812; Granger & Cyr, 2001, Plant Cell Repo. 20: 227-234), temporally regulated, spatially regulated, tissue-specific, and spatio-temporally regulated (Kuhlemeier et al. 1987, Ann. Rev. Plant Physiol. Plant Mol. Biol. 38: 221-257). Promoters from bacteria, fungi, viruses and plants have been used to control gene expression in plant cells.

Promoters consist of several regions that are necessary for full function of the promoter. Some of these regions are modular, in other words they can be used in isolation to confer promoter activity or they may be assembled with other elements to construct new promoters. The first of these promoter regions lies immediately upstream of the coding sequence and forms the “core promoter region” containing consensus sequences, normally 20-70 base pairs immediately upstream of the coding sequence. The core promoter region contains a TATA box and often an initiator element as well as the initiation site. The precise length of the core promoter region is not fixed but is usually well recognizable. Such a region is normally present, with some variation, in most promoters. The base sequences lying between the various well-characterized elements appear to be of lesser importance. The core promoter region is often referred to as a minimal promoter region because it is functional on its own to promote a basal level of transcription.

The presence of the core promoter region defines a sequence as being a promoter: if the region is absent, the promoter is non-functional. The core region acts to attract the general transcription machinery to the promoter for transcription initiation. However, the core promoter region is insufficient to provide full promoter activity. A series of regulatory sequences, often upstream of the core, constitute the remainder of the promoter. The regulatory sequences determine expression level, the spatial and temporal pattern of expression and, for a subset of promoters, expression under inductive conditions (regulation by external factors such as light, temperature, chemicals and hormones). Regulatory sequences may be short regions of DNA sequence 6-100 base pairs that define the binding sites for trans-acting factors, such as transcription factors. Regulatory sequences may also be enhancers, longer regions of DNA sequence that can act from a distance from the core promoter region, sometimes over several kilobases from the core region. Regulatory sequence activity may be influenced by trans-acting factors including general transcription machinery, transcription factors and chromatin assembly factors.

Frequently, it is desirable to have tissue-specific expression of a gene of interest in a plant. Tissue-specific promoters promote expression exclusively in one set of tissues without expression throughout the plant; tissue-preferred promoters drive expression at a higher level in a subset of tissues with significantly less expression in the other tissues of the plant. For example, one may desire to express a value-added product only in corn seed but not in the remainder of the plant. Another example is the production of male sterility by tissue-specific ablation. In this case, expression is isolated to the root, more specifically the root cap. Many aspects of agricultural biotechnology use and require tissue-specific expression.

The maize root cap may consist up to as many as 10,000 cells. These cap cells originate in the meristem, which functions only to produce new caps cells. The meristem has a cell cycle time of approximately 12 hours. After a cell is made in the meristem, it passes down the root cap and is eventually lysed and becomes mucigel. The journey of a Maize root cap cell from the meristem to the mucigel takes from five to eight days.

Depending on the anatomy of the root tip, meristems have been classified as open or closed. Maize has a closed root system, which means that there is convergence of cell files at the root apex that makes it clear to distinguish the different sections of the root. This compares to an open root system that has no discrete boundary between the root proper and the root cap that makes it difficult to trace cell files ((Lim The Plant Cell 12: 1307-1318 (2000)). The root systems of maize are similar to that of grasses, consisting of distinct types of roots that are formed at different stages and location during development. During embryogenesis, the primary and lateral roots are formed. In postembryonic development, crown roots and brace/prop roots arise from stem tissue.

This provides an important example of a need for promoters in the expression of selected genes in plant roots. The plant root consists of many cell types such as epidermal, root cap, columella, cortex, pericycle, vascular and root hair forming trichoblasts, organized into tissues or regions of the root, for example, the root tip, root epidermis, meristematic zone, primary root, lateral root, root hair, and vascular tissue. Promoters isolated as root-specific or root-preferred can be biased towards promotion of expression in one or a few of these cell types. This cell-specific activity can be useful for specific applications such as regulating meristematic activity in only the meristematic cell zone or expression of a nematicidal gene in only the cell types that are contacted by the nematode pest. In other cases, broader cell-type specificity may be desired to express genes of interest throughout the root tissue. This may be useful in expressing an insecticidal gene to control an insect pest that feeds on plant roots, for instance corn rootworm (Diabrotica spp.). Root cap specificity may be accomplished with a single root-specific promoter with broad cell-type specificity or by using two or more root-specific or root-preferred promoters of different cell-type specificities for expression. A limited number of examples of root-preferred and root-specific promoters have been described. These include the RB7 promoter from Nicotiana tabacum (U.S. Pat. Nos. 5,459,252 and 5,750,386); the ARSK1 promoter from Arabidopsis thaliana (Hwang and Goodman (1995) Plant J 8:37-43), the MR7 promoter from Zea mays (U.S. Pat. No. 5,837,848), the ZRP2 promoter of Zea mays (U.S. Pat. No. 5,633,363), and the MTL promoter from Zea mays (U.S. Pat. Nos. 5,466,785 and 6,018,099). Many of these examples disclose promoters with expression patterns confined to a limited number of root tissues. Others fail to provide the root-specificity needed for expression of selected genes. Thus, there is a need in the art for isolation and characterization of new root promoters to obtain those of different breadth, expression level and specificity of cell-type expression for root-specific and root-preferred expression, particularly for root cap specific expression.

### SUMMARY OF THE INVENTION

Within the invention, compositions and methods for directing root cap specific expression in transgenic plants are provided. In particular, a novel nucleic acid molecule isolated from Zea mays, that drives expression of heterologous genes in a root cap specific manner in plants, are provided. The invention is further drawn to expression cassettes and vectors comprising the novel nucleic acid molecule of the invention operably linked to heterologous coding sequences. The invention is still further drawn to transgenic plants comprising the expression cassettes of the invention. The invention also provides methods for specifically expressing a heterologous coding sequence in transgenic plant roots, for isolating a root-specific cDNA, for isolating a nucleic acid molecule useful for directing root-specific expression and for isolating a root-specific promoter. The invention further provides primers and nucleic acid probes to identify related nucleotide sequences from other plant genomes that direct root cap-specific or root cap-preferred transcription.

According to one aspect, the invention provides an isolated nucleic acid molecule which codes for a promoter capable of directing root cap-specific transcription in a plant, wherein the nucleotide sequence of the promoter comprises a nucleotide sequence set forth in SEQ ID NO: 1.

According to one aspect, the invention provides an isolated nucleic acid molecule which codes for a promoter capable of directing root cap-specific transcription in a plant, wherein the nucleotide sequence of the promoter comprises a nucleotide sequence set forth in SEQ ID NO: 2. SEQ ID NO: 2 is a truncated version of SEQ ID NO: 1.

The invention also provides an expression cassette comprising the nucleic acid molecule of the invention operably linked to a heterologous coding sequence. In one aspect, the expression cassette comprises a heterologous coding sequence selected from the group consisting of insecticidal coding sequences, nematicidal coding sequences, herbicide-tolerance coding sequences, anti-microbial coding sequences, anti-fungal coding sequences, anti-viral coding sequences, abiotic stress tolerance coding sequences, nutritional quality coding sequences, visible marker coding sequences and selectable marker coding sequences. In another embodiment, the expression cassette comprises an insecticidal coding sequence that encodes a toxin active against a coleopteran pest. In an aspect of this embodiment, the coleopteran pest is a species in the genus Diabrotica. In yet another embodiment, the expression cassette comprises an abiotic stress tolerance coding sequence including but not limited to drought stress, nutrient stress, salt stress, water stress and heavy metal stress. In still another embodiment, the expression cassette comprises a visible marker coding sequence including but not limited to green fluorescent protein (GFP), β-glucuronidase (GUS), and luciferase (LUC). In yet another embodiment, the expression cassette comprises a selectable marker coding sequence including but not limited to phosphomannose isomerase (PMI), an antibiotic resistance gene such as hygromycin, kanamycin and the like, a herbicide tolerance gene such as phosphinothricin (PAT), barnase (BAR), EPSPS, GAT and the like.

The invention also provides a recombinant vector comprising the expression cassette of the invention. In an aspect of this embodiment, the recombinant vector is a plasmid.

Further, the invention provides a transgenic non-human host cell comprising the expression cassette of the invention. A transgenic host cell according to this aspect of the invention is preferably a plant cell. Even further, the invention provides a transgenic plant comprising such a transgenic plant cell. A transgenic plant according to this aspect of the invention may be sorghum, wheat, sunflower, tomato, cole crops, cotton, rice, soybean, sugar beet, sugarcane, tobacco, barley, oilseed rape and maize, preferably maize. Still further, the invention provides transgenic seed from the group of transgenic plants consisting of sorghum, wheat, sunflower, tomato, cole crops, cotton, rice, soybean, sugar beet, sugarcane, tobacco, barley, oilseed rape and maize. In an embodiment of the invention, the transgenic seed is from a transgenic maize plant.

In another aspect, the invention provides a method of specifically expressing a heterologous coding sequence in transgenic plant roots under transcriptional control of a nucleic acid molecule of the invention, comprising: (a) transforming plant cells with a vector wherein the vector comprises the nucleic acid molecule of the invention operably linked to a heterologous coding sequence, (b) growing the transgenic plant cells comprising the vector, and (c) producing transgenic plants from the transformed plant cells wherein the heterologous coding sequence is specifically expressed in plant roots under control of a nucleic acid molecule of the invention. In one embodiment of this aspect, the transgenic plant is a maize plant or a rice plant. In another embodiment of this aspect, the heterologous coding sequence is selected from the group consisting of insecticidal coding sequences, nematicidal coding sequences, herbicide tolerance coding sequences, anti-microbial coding sequences, anti-fungal coding sequences, anti-viral coding sequences, abiotic stress tolerance coding sequences, nutritional quality coding sequences, visible marker coding sequences and selectable marker coding sequences. In yet another embodiment, the invention provides transgenic plants produced according to this aspect. In another embodiment the transgenic plants are maize plants or rice plants.

Also provided by the invention are nucleic acid primers comprising at least 16 contiguous nucleotides of any one of SEQ ID NOS: 1-2. These primers are useful in detecting the presence of the promoters. Examples of such primers are set forth in SEQ ID NOS: 7-10.

### BRIEF DESCRIPTION OF THE SEQUENCES IN THE SEQUENCE LISTING

SEQ ID NO: 1 is the nucleotide sequence of the ZmRCP1-1 promoter.
SEQ ID NO: 2 is the nucleotide sequence of the ZmRCP1-2 promoter.
SEQ ID NO: 3 is the nucleotide sequence of the ZmRCP1 CDS
SEQ ID NO: 4 is the nucleotide sequence of the ZmRCP1 mRNA.
SEQ ID NO: 5 is the nucleotide sequence of the pNOV6901 vector.
SEQ ID NO: 6 is the nucleotide sequence of the pSYN15605 vector.
SEQ ID NO: 7 is the nucleotide sequence of the pSYN15861 vector.
SEQ ID NO: 8 is the nucleotide sequence of the pSYN15888 vector.
SEQ ID NO: 9 is the nucleotide sequence of the ZmRCP1-1 promoter forward primer.
SEQ ID NO: 10 is the nucleotide sequence of the ZmRCP1-1 promoter reverse primer.
SEQ ID NO: 11 is the nucleotide sequence of the ZmRCP1-2 promoter forward primer.
SEQ ID NO: 12 is the nucleotide sequence of the ZmRCP1-2 promoter reverse primer.
SEQ ID NO: 13 is the nucleotide sequence of the ZmRCP mutation 1 primer.
SEQ ID NO: 14 is the nucleotide sequence of the ZmRCP mutation 2 primer.
SEQ ID NO: 15 is the nucleotide sequence of the ZmRCP mutation 3 primer.
SEQ ID NO: 16 is the nucleotide sequence of the ZmRCP mutation 4 primer.
SEQ ID NO: 17 is the nucleotide sequence of the ZmRCP mutation 5 primer.
SEQ ID NO: 18 is the nucleotide sequence of the ZmRCP mutation 6 primer.
SEQ ID NO: 19 is the nucleotide sequence of the ZmRCP1 terminator forward primer.
SEQ ID NO: 20 is the nucleotide sequence of the ZmRCP1 terminator reverse primer.
SEQ ID NO: 21 is the nucleotide sequence of the pSYN15670 vector.

### DEFINITIONS

“Antisense inhibition” refers to the production of antisense RNA transcripts capable of suppressing the expression of protein from an endogenous gene or a transgene.

“Chimeric” is used to indicate that a DNA sequence, such as a vector or a gene, is comprised of two or more DNA sequences of distinct origin that are fused together by recombinant DNA techniques resulting in a DNA sequence, which does not occur naturally.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

“Constitutive promoter” refers to a promoter that is able to express the gene that it controls in all or nearly all of the plant tissues during all or nearly all developmental-stages of the plant, thereby generating “constitutive expression” of the gene.

“Co-suppression” and “sense suppression” refer to the production of sense RNA transcripts capable of suppressing the expression of identical or substantially identical transgene or endogenous genes (U.S. Pat. No. 5,231,020).

“Contiguous” is used herein to mean nucleic acid sequences that are immediately preceding or following one another.

“Corn rootworm” or “corn rootworms”, as used herein, refer to insects of the genus Diabrotica, including the southern corn rootworm, the northern corn rootworm, the western corn rootworm, and the Mexican corn rootworm either in the larval or adult stage, preferably in the larval stage. The root cap-specific promoter of the invention are used to express corn rootworm toxins in the roots of transgenic plants thus protecting fields of transgenic plants from corn rootworm damage. The term “corn rootworm” and Diabrotica are herein used interchangeably.

“Expression” refers to the transcription and stable accumulation of mRNA. Expression may also refer to the production of protein.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components.

The “expression pattern” of a promoter (with or without an enhancer) is the pattern of expression level that shows where in the plant and in what developmental stage the promoter initiates transcription. Expression patterns of a set of promoters are said to be complementary when the expression pattern of one promoter shows little overlap with the expression pattern of the other promoter.

“Gene” refers to a nucleic acid fragment that expresses mRNA, functional RNA, or specific protein, including regulatory sequences. The term “Native gene” refers to a gene as found in nature. The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes. (English, et al., 1996, Plant Cell 8:179-1881). Gene silencing includes virus-induced gene silencing (Ruiz et al., 1998, Plant Cell 10:937-946).

“Genetically stable” and “heritable” refer to chromosomally-integrated genetic elements that are stably maintained in the plant and stably inherited by progeny through successive generations.

“Heterologous DNA Sequence” is a DNA sequence not naturally associated with a host cell into which it is introduced, including non-naturally occurring multiple copies of a naturally occurring DNA sequence.

“Inducible promoter” refers to those regulated promoters that can be turned on in one or more cell types by an external stimulus, such as a chemical, light, hormone, stress, or a pathogen.

“Insecticidal” is defined as a toxic biological activity capable of controlling insects, preferably by killing them.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency. (Turner et al., 1995, Molecular Biotechnology, 3:225).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989, Plant Cell, 1:671-680).

The term “nucleic acid” refers to a polynucleotide of high molecular weight which can be single-stranded or double-stranded, composed of monomers (nucleotides) containing a sugar, phosphate and a base which is either a purine or pyrimidine. A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers.

The terms “open reading frame” and “ORF” refer to the amino acid sequence encoded between translation initiation and termination codons of a coding sequence. The terms “initiation codon” and “termination codon” refer to a unit of three adjacent nucleotides ('codon') in a coding sequence that specifies initiation and chain termination, respectively, of protein synthesis (mRNA translation).

“Operably-linked” and “Operatively-linked” refer to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

“Overexpression” refers to the level of expression in transgenic organisms that exceeds levels of expression in normal or untransformed organisms.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

“Preferred expression” is the expression of gene products that are preferably expressed at a higher level in one or a few plant tissues (spatial limitation) and/or to one or a few plant developmental stages (temporal limitation) while in other tissues/developmental stages there is a relatively low level of expression.

“Primary transformant” and “T0 generation” refer to transgenic plants that are of the same genetic generation as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The terms “protein,” “peptide” and “polypeptide” are used interchangeably herein.

A “promoter” is an untranslated DNA sequence typically upstream of a coding region that contains the binding site for RNA polymerase and initiates transcription of the DNA. The promoter region may also include other elements that act as regulators of gene expression. “Promoter regulatory sequences” consist of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Promoters may be derived in their entirety from a native gene, or be composed of different elements derived from different promoters found in nature, or even be comprised of synthetic DNA segments. A “minimal or core promoter” is a promoter consisting only of all basal elements needed for transcription initiation, such as a TATA-box and/or initiator.

“Reference sequence” as used herein is defined as a sequence that is used as a basis for sequence comparison. A reference sequence may be a subset or the entirety of a specified sequence; for example, as a fragment of a full-length cDNA or gene sequence, or the full-length cDNA or gene sequence.

“Regulated promoter” refers to promoters that direct gene expression not constitutively, but in a temporally- and/or spatially-regulated manner, and include both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived by posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA. A “functional RNA” refers to an antisense RNA, ribozyme, or other RNA that is not translated, but participates in a reaction or process as a RNA.

The term “root” refers to the base structure of a plant. A root is the usually underground part of a seed plant body that usually originates from the hypocotyls. It functions as an organ of absorption, aeration, and food storage or as a means of anchorage and support. A root differs from a stem especially in lacking nodes, buds, and leaves. Maize plants have three types of roots: seminal, adventitious and brace. Seminal roots develop from radicle and persist for long period. Adventitious roots are fibrous roots developing from the lower nodes of stem below ground level are the effective and active roots of plant. Brace or prop roots are produced by the lower two nodes of the stem.

The term “root cap” refers to a thimblelike mass of parenchyma cells that covers and protects the growing root tip as it penetrates the soil. The root cap is pushed forward as the root tip grows longer. The cells on the periphery of the root cap are sloughed as the root cap is pushed forward and new cells are added by the apical meristem. The root cap protects the apical meristem, aids the root as it penetrates the soil, and plays an important role in controlling the response of the root to gravity (gravitropism).

A “selectable marker gene” refers to a gene whose expression in a plant cell gives the cell a selective advantage. The selective advantage possessed by the cells transformed with the selectable marker gene may be due to their ability to grow in presence of a negative selective agent, such as an antibiotic or a herbicide, compared to the ability to grow of non-transformed cells. The selective advantage possessed by the transformed cells may also be due to their enhanced capacity, relative to non-transformed cells, to utilize an added compound as a nutrient, growth factor or energy source. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell, typically from a transgenic plant.

“Specific expression” is the expression of gene products that is limited to one or a few plant tissues (spatial limitation) and/or to one or a few plant developmental stages (temporal limitation).

Substantially identical: the phrase “substantially identical,” in the context of two nucleic acid or protein sequences, refers to two or more sequences or subsequences that have at least 60%, preferably 80%, more preferably 90, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In an embodiment of the invention, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Those of skill in the art understand that to avoid a high similarity to a reference sequence due to inclusion of gaps in the polynucleotide sequence a gap penalty is typically introduced and is subtracted from the number of matches.

Optimal alignment of sequences for comparison can be conducted, e.g., by the local homology algorithm of Smith & Waterman, 1981, Adv. Appl. Math. 2: 482, by the homology alignment algorithm of Needleman & Wunsch, 1970, J. Mol. Biol. 48: 443, by the search for similarity method of Pearson & Lipman, 1988, Proc. Nat'l. Acad. Sci. 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., 1990, J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, 1989, Proc. Natl. Acad. Sci. 89: 10915).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

For purposes of the invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

Another indication that two nucleic acid sequences are substantially identical is that the two molecules hybridize to each other under stringent hybridization conditions. The phrase “hybridizing specifically to” refers to the binding, duplexing, or hybridizing of a molecule only to a particular nucleotide sequence under stringent hybridization conditions when that sequence is present in a complex mixture (e.g., total cellular) of DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2, “Overview of principles of hybridization and the strategy of nucleic acid probe assays”, Elsevier, New York. Generally, high stringency hybridization and wash conditions are selected to be about 5° C. lower than the thermal melting point (Tₘ) for the specific sequence at a defined ionic strength and pH. Typically, under high stringency conditions a probe will hybridize to its target subsequence, but to no other sequences.

The Tₘ is the temperature (under defined ionic strength and pH) at which 50% of the target sequence hybridizes to a perfectly matched probe. Very high stringency conditions are selected to be equal to the Tᵣ, for a particular probe. An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.1 5M NaCl at 72° C. for about 15 minutes. An example of high stringency wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, for example, when a copy of a nucleic acid is created using the maximum codon degeneracy permitted by the genetic code.

Low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium. citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 0% formamide, 1 M NaCl, 1% SDS at 37° C., and a wash in 0.1×SSC at 60 to 65° C.

The following are examples of sets of hybridization/wash conditions that may be used to clone homologous nucleotide sequences that are substantially identical to reference nucleotide sequences of the invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Specificity is typically the function of post-hybridization washes, the critical factors being the ionic strength and temperature of the final wash solution. For DNA-DNA hybrids, the Tₘ can be approximated from the equation of Meinkoth and Wahl Anal. Biochem. 138:267-284 (1984); TM 81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The TM is the temperature (under defined ionic strength and pH) at which 50% of a complementary target sequence hybridizes to a perfectly matched probe. T is reduced by about 1° C. for each 1% of mismatching; thus, TM, hybridization, and/or wash conditions can be adjusted to hybridize to sequences of the desired identity. For example, if sequences with >90% identity are sought, the Tₘ can be decreased 10° C. Generally, high stringency conditions are selected to be about 19° C. lower than the thermal melting point (Tₘ) for the specific sequence and its complement at a defined ionic strength and pH. However, very high stringency conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part 1, Chapter 2 (Elsevier, New York); and Ausubel et al., eds. (1995) Current Protocols in Molecular Biology, Chapter 2 (Greene Publishing and Wiley—Interscience, New York). See Sambrook et al. (1989) Molecular Cloning: A Laborator Manual (2d ed., Cold Spring Harbor Laboratory Press, Plainview, N.Y.).

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

“Tissue-specific promoter” refers to regulated promoters that are not expressed in all plant cells but only in one or more cell types in specific organs (such as leaves, roots or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

“Transactivating gene” refers to a gene encoding a transactivating protein. It can encode a transcription factor. It can be a natural gene, for example, a plant transcriptional activator, or a chimeric gene, for example, when plant regulatory sequences are operably-linked to the open reading frame of a transcription factor from another organism. “Transactivating genes” may be chromosomally integrated or transiently expressed. “Trans-activation” refers to switching on of gene by the expression of another (regulatory) gene in trans.

A “transcriptional cassette” will comprise in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source.

The “transcription initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

The term “transformation” refers to the transfer of a nucleic acid fragment into the genome of a host cell, resulting in genetically stable inheritance. “Transiently transformed” refers to cells in which transgenes and foreign DNA have been introduced (for example, by such methods as Agrobacterium-mediated transformation or biolistic bombardment), but not selected for stable maintenance. “Stably transformed” refers to cells that have been selected and regenerated on a selection media following transformation.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

“Transient expression” refers to expression in cells in which a virus or a transgene is introduced by viral infection or by such methods as Agrobacterium-mediated transformation, electroporation, or biolistic bombardment, but not selected for its stable maintenance.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eucaryotic (e.g. higher plant, mammalian, yeast or fungal cells).

“Visible marker” refers to a gene whose expression does not confer an advantage to a transformed cell but can be made detectable or visible. Examples of visible markers include but are not limited to β-glucuronidase (GUS), luciferase (LUC) and green fluorescent protein (GFP).

“Wild-type” refers to the normal gene, virus, or organism found in nature without any known mutation.

### DETAILED DESCRIPTION OF THE INVENTION

### Identification of Root-Specific Genes, Promoters and Homologues.

In many instances, it is desirable to spatially regulate the expression of a transgene so as to be expressed only in plant root tissues. A promoter capable of directing expression in a specific or preferential manner can most expeditiously accomplish this spatial regulation.

The invention provides isolated nucleic acid molecules having a nucleotide sequence that directs root-specific transcription in a plant. Root-specific promoters are isolated by identifying genes that are specifically expressed in root tissues of a target plant and subsequently isolating the regulatory sequences of these genes.

It is also clear to one skilled in the art that mutations, insertions, deletions and/or substitutions of one or more nucleotides can be introduced into the nucleotide sequence of SEQ ID NO: 1 using methods known in the art. In addition, shuffling the sequences of the invention can provide new and varied nucleotide sequences.

To test for a function of variant DNA sequences according to the invention, such as deletion fragments of SEQ ID NO: 1, the sequence of interest is operably linked to a selectable or visible marker gene and expression of the marker gene is tested in transient expression assays with isolated root tissue or cells or by stable transformation into plants. It is known to the skilled artisan that DNA sequences capable of driving expression of an associated coding sequence are built in a modular way. Accordingly, expression levels from shorter DNA fragments may be different than the one from the longest fragment and may be different from each other. For example, deletion of a down-regulating upstream element will lead to an increase in the expression levels of the associated coding sequence while deletion of an up-regulating element will decrease the expression levels of the associated coding sequence. It is also known to the skilled artisan that deletion of development-specific or a tissue-specific elements will lead to a temporally or spatially altered expression profile of the associated coding sequence.

In another embodiment of the invention, DNA and genomic DNA sequences homologous to SEQ ID NO: 1 may be isolated from other maize germplasm using either hybridization or PCR techniques well known in the art. The isolated sequences may be identical to SEQ ID NO: 1 or they may be substantially identical to SEQ ID NO: 1. It is not necessary for the sequences obtained from other maize germplasm to contain identical nucleotide sequences to be functionally identical to the sequences disclosed herein. Some nucleotide deletions, additions, and replacements may have no impact or only a minor impact on gene expression. One aspect is an isolated nucleic acid molecule, according to the invention, comprising a nucleotide sequence that has at least 70% identity to the nucleotide sequence set forth in SEQ ID NO: 1. Another aspect is an isolated nucleic acid molecule comprises a nucleotide sequence that has at least 80% identity to the nucleotide sequence set forth in SEQ ID NOS: 1. Another aspect is an isolated nucleic acid molecule comprising a nucleotide sequence that has at least 90% identity to any one of the nucleotide sequences set forth in SEQ ID NO: 1. Another aspect is isolated nucleic acid molecule comprising a nucleotide sequence that has at least 95% identity to the nucleotide sequence set forth in SEQ ID NO: 1. Another aspect is an isolated nucleic acid molecule comprising a nucleotide sequence that has at least 99% identity to the nucleotide sequences set forth in SEQ ID NO: 1. Another aspect is an isolated nucleic acid molecule comprising any one of the nucleotide sequences set forth is SEQ ID NO: 1.

In another embodiment of the invention, cDNA and genomic DNA sequences may be cloned from other plants that represent homologues of the root-specific maize genes and promoters. These homologues allow one to obtain additional root-specific promoters useful for the regulation of multiple genes in the root. Hybridization using the maize cDNA and genomic sequences or portions thereof is used to screen for homologous or substantially identical sequences in other plant genomes. These sequences may comprise only a subset of the nucleotides of SEQ ID NOS: 1-2. A preferable length of homology is 20 base pairs (bp) in length, more preferably, 50 bp in length, and most preferably at least 100 bp in length. In one embodiment of the invention, a hybridization probe is prepared from any one SEQ ID NOS: 1-2 or portions thereof. Hybridization of such sequences may be carried out under high stringency conditions. Alternatively, low or moderate stringency conditions can be used to allow some mismatching in sequences so that lower degrees of similarity are detected (heterologous probing). Generally, a probe is less than about 1000 nucleotides in length, preferably less than 500 nucleotides in length.

In another embodiment of the invention, cDNA and genomic sequences are isolated by preparing primers comprising sequences within any one of SEQ ID NOS: 1-2. The primers may be used in a PCR reaction with cDNA or genomic DNA from a plant to obtain homologous sequences or sequences with substantial identity to any one of SEQ ID NOS: 1-2.

### Construction of Expression Cassettes

Expression cassettes are constructed comprising the 5′ flanking sequences of the root-specific genomic clones. In one embodiment of the invention, the promoter region utilized in each expression cassette comprises the 5′ flanking region up to and including the start of translation. The start of translation is denoted by the first ATG of the open reading frame (ORF) found in the cDNA and the homologous genomic sequence. Thus, the promoter region may include 5′ untranslated leader sequence as well as the transcriptional start site, core promoter and additional regulatory elements. In another embodiment of the invention, expression cassettes are constructed comprising the 5′ flanking sequence of the root-specific genomic clones up to and including the transcriptional initiation site. The transcriptional initiation site may be defined by the first nucleotide of the longest cDNA clone obtained. Additionally, the transcriptional initiation site may be further defined by use of techniques well known in the art including RACE PCR, RNase protection mapping and primer extension analysis.

The expression cassettes may further comprise a transcriptional terminator, downstream (3′) to the promoter. A variety of transcriptional terminators are available for use in expression cassettes. The transcriptional terminator is responsible for the termination of transcription beyond the transgene and correct mRNA polyadenylation of the mRNA transcript. Appropriate transcriptional terminators are those that are known to function in plants and include the CaMV 35S terminator, the tml terminator, the nopaline synthase terminator, the pea rbcS E9 terminator and the ZmRCP1 terminator. These can be used in both monocotyledons and dicotyledons. In addition, a gene's native transcription terminator may be used. For example, the 3′ flanking sequence comprising genomic sequence 3′ to the region homologous to a root-specific cDNA clone may be used.

In an embodiment of the invention a heterologous coding sequence, for example, an insecticidal coding sequence, a visible marker coding sequence, or a selectable marker coding sequence, is cloned between a promoter of the invention and transcriptional terminator whereby the heterologous coding sequence is operatively linked to the promoter and the transcriptional terminator is operatively linked to the heterologous coding sequence. Examples of visible markers useful for the invention include, but are not limited to, β-glucuronidase (GUS), Chloramphenicol Acetyl Transferase (CAT), Luciferase (LUC) and proteins with fluorescent properties, such as Green Fluorescent Protein (GFP) from Aequora victoria. In principle, many more proteins are suitable for this purpose, provided the protein does not interfere with essential plant functions. Further examples of heterologous coding sequences useful for the invention include, but are not limited to, antibiotic resistance, virus resistance, insect resistance, disease resistance, or resistance to other pests, herbicide tolerance, improved nutritional value, improved performance in an industrial process or altered reproductive capability. In an aspect of this embodiment of the invention, a gene encoding for resistance to insects that feed on the roots of the plant is cloned between the promoter and terminator. In another embodiment of the invention a sequence encoding a functional RNA such as antisense RNA, a sense RNA for sense-suppression, or a double stranded RNA may also be cloned between the promoter and transcriptional terminator.

In another embodiment, the promoter can be used to improve root development, water and nutrient absorption and utilization, and consequently stress tolerance through a transgenic approach.

Numerous sequences have been found to enhance gene expression from within the transcriptional unit and these sequences can be used in conjunction with the promoters of this invention to increase their expression in transgenic plants. Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze1 gene had a similar effect in enhancing expression. Intron sequences have been routinely incorporated into plant transformation vectors, typically within the non-translated leader. A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Sarnow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

### Plant Transformation Methods Useful for the Invention

Numerous transformation vectors available for plant transformation are known to those of ordinary skill in the plant transformation art, and the nucleic acid molecules of the invention can be used in conjunction with any such vectors. The selection of vector will depend upon the preferred transformation technique and the target plant species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet. 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

### Vectors Suitable for Agrobacterium Transformation

Many vectors are available for transformation using Agrobacterium tumefaciens.

These typically carry at least one T-DNA border sequence and include vectors such as pBIN19 (Bevan, Nucl. Acids Res. (1984)). Below, the construction of a typical vector suitable for Agrobacterium transformation is described.

### Vectors Suitable for Non Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed.

### Transformation Methods Useful for the Invention

Once a nucleic acid molecule of the invention has been cloned into an expression cassette, it is transformed into a plant cell. The receptor and target expression cassettes of the invention can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Plants transformed in accordance with the invention may be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis thaliana, and woody plants such as coniferous and deciduous trees, especially maize, wheat, or rice.

Once an expression cassette is transformed into a particular plant species, the expression cassette may be propagated in that species or moved into other varieties of the same species, particularly including commercial varieties, using traditional breeding techniques.

### Transformation of Dicotyledons

Transformation techniques for dicotyledons are well known in the art and include both Agrobacterium-based and non-Agrobacterium based techniques. Non-Agrobacterium techniques involve the uptake of exogenous genetic material directly by protoplasts or cells. This can be accomplished by particle bombardment-mediated delivery, microinjection, or PEG or electroporation mediated uptake. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a tri-parental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the vector can surround the target cell so that the vector is carried into the cell by the wake of the particle. Biologically active particles (e.g., dried yeast cells, dried bacterium or a bacteriophage, each containing DNA sought to be introduced) can also be propelled into plant cell tissue.

### Transformation of Monocotyledons

Transformation of most monocotyledon species has now also become routine. Preferred techniques include direct gene transfer via Agrobacterium into immature embryos, protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11: 1553-1558 (1993)) and Weeks et al. (Plant Physiol. 102: 1077-1084 (1993)) using particle bombardment of immature embryos and immature embryo-derived callus. One technique for wheat transformation involves the transformation of wheat by particle bombardment of immature embryos and includes either a high sucrose or a high maltose step prior to gene delivery. Prior to bombardment, embryos that are 0.75-1 mm in length are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 h and are then bombarded. Twenty embryos per target plate are typical, although not critical. An appropriate gene-carrying plasmid (such as pCIB3064 or pSG35) is precipitated onto micrometer size gold particles using standard procedures. Each plate of embryos is shot with the DuPont Biolistics® helium device using a burst pressure of ˜1000 psi using a standard 80 mesh screen. After bombardment, the embryos are placed back into the dark to recover for about 24 h (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described in WO 94/00977 and U.S. Pat. No. 5,591,616, both of which are incorporated herein by reference. A preferred method of maize transformation is described in Negrotto et al., (Plant Cell Reports 19: 798-803 (2000)), incorporated herein by reference.

### Analysis of Promoter Activity

Several methods are available to assess promoter activity. Expression cassettes are constructed with a visible marker, as described above. Transient transformation methods are used to assess promoter activity. Using transformation methods such as microprojectile bombardment, Agrobacterium transformation or protoplast transformation, expression cassettes are delivered to plant cells or tissues. Reporter gene activity, such as β-glucuronidase activity, luciferase activity or GFP fluorescence is monitored after transformation over time, for example 2 hours, 5 hours, 8 hours, 16 hours, 24 hours, 36 hours, 48 hours and 72 hours after DNA delivery using methods well known in the art. Reporter gene activity may be monitored by enzymatic activity, by staining cells or tissue with substrate for the enzyme encoded by the reporter gene or by direct visualization under an appropriate wavelength of light. Full-length promoter sequences, deletions and mutations of the promoter sequence may be assayed and their expression levels compared. Additionally, RNA levels may be measured using methods well known in the art such as Northern blotting, competitive reverse transcriptase PCR and RNAse protection assays. These assays measure the level of expression of a promoter by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore the steady state level is the product of synthesis rates and degradation rates. The rate of degradation can however be considered to proceed at a fixed rate when the transcribed sequences are identical, and thus this value can serve as a measure of synthesis rates.

Further confirmation of promoter activity is obtained by stable transformation of the promoter in an expression cassette comprising a visible marker or gene of interest into a plant as described above. Using the various methods described above such as enzymatic activity assays, RNA analysis and protein assays as described supra, promoter activity is monitored over development, and additionally by monitoring expression in different tissues in the primary transformants and through subsequent generations of transgenic plants.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described by Ausubel (ed.), Current Protocols in Molecular Biology, John Wiley and Sons, Inc. (1994); J. Sambrook, et al., Molecular Cloning: A Laboratory Manual, 3d Ed., Cold Spring Harbor, N.Y.: Cold Spring Harbor Laboratory Press (2001); and by T. J. Silhavy, M. L. Berman, and L. W. Enquist, Experiments with Gene Fusions, Cold Spring Harbor Laboratory, Cold Spring Harbor, N.Y. (1984).

### Example 1

### Construction of Root Cap-Specific Expression Cassettes

### Entry Vectors

The first step in construction of expression cassettes was the cloning of the promoter into an entry vector. PCR primers were designed to amplify the promoter and terminator from maize line B73. These isolated nucleotide sequences were TOPO cloned and sequenced. The promoter corresponding to this sequence was designated as Maize Root Cap-Specific 1 promoter or ZmRCP1-1 promoter. The terminator corresponding to this sequence was designated as Maize Root Cap-Specific 1 terminator or ZmRCP1-terminator.

The ZmRCP1-1 promoter was amplified from maize genomic DNA (B73) template in a 50 μL Extensor (ABgene) DNA polymerase reaction containing 10 μg gDNA, 5 μL 10× Extensor Buffer 1, 2.0 μL 10 mM dNTP mix, 1.0 μL of 20 μM prRCP forward—SEQ ID NO: 9 (5′GCTAGCCTCGAGGGACCCAACAATTTGCCACAAACTGG-3′), 1.0 μL of 20 μM RCP P2 reverse—SEQ ID NO: 10 (5′-GCTAGCGGATCCGGCGCCGCCGGGATAGAAGTCGCACAC-3′), 10.0 μL 5×Q solution and 1 μl Extensor DNA polymerase. The thermocycling program was 95° C. for 2 minutes followed by 40 cycles of 95° C. for 30 seconds, 50° C. for 60 seconds and 68° C. for 5 minutes. The final extension step was 68° C. for 15 minutes. The 1.5 kb reaction product was gel-purified on 1% TBE agarose and the DNA was extracted using Qiaprep DNA extraction method. The DNA was cloned into the pCR4-Blunt-TOPO vector.

The ZmRCP1-1 promoter was modified in a series of QuikChange reactions to add STOP codons to open reading frames (ORF) and correct point mutations created during amplification using the Stratagene QuikChange Multi-site mutagenesis kit. The 25 μL reaction contained 1 μL pCR4-Blunt-TOPO-prZmRCP, 2.5 μL 10× QuikChange buffer, 1 μL QuikChange dNTP mix, 0.75 μL Quik solution, 1 μL QuikChange DNA polymerase and 1 μL of 20 μM of at least one of the following oligonucleotides:

SEQ ID NO: 13
prRCP mut1
(5′- GCGGCGGCGGCGTAGTTGCAACCCGCATC -3′),
SEQ ID NO: 14
prRCP mut2
(5′- GCAGTGTGCGACTTGAATCCCGGCGGCGCC -3′),
SEQ ID NO: 15
prRCP mut3
(5′- CTACTCCATGCTAAAGCTGTAGAGCCGAG -3′),
SEQ ID NO: 16
prRCP mut4
(5′- CCTTTATCAATTTGCCTCGATCTCCATAG -3′) ,
SEQ ID NO: 17
prRCP mut5
(5′- GAAACTTGTTTGTTGTTATTAATTTTCAAC -3′),
and
SEQ ID NO: 18
prRCP mut6
(5′- GCACCAACATCAAGAGCAACAAGACCACC -3′).

The thermocycling program was 95° C. for 1 minute followed by 35 cycles of 95° C. for 1 minute, 55° C. for 1 minute and 65° C. for 15 minutes. The product was processed as described by the manufacturer (Stratagene) and completely sequenced.

The corrected ZmRCP1-1 promoter was cut from the TOPO vector by XhoI/BamHI and ligated to a similarly cut pNOV6901, SEQ ID NO: 5.

The ZmRCP terminus was amplified from maize genomic DNA (B73) template in a 50 μL Expand (Roche) DNA polymerase reaction containing 10 μg gDNA, 5 μL 10× Expand High Fidelity buffer with MgCl₂, 1.0 μL 10 mM dNTP mix, 2.0 μl of 2% DMSO, 2.0 μL of 20 μM tRCP forward—SEQ ID NO: 19 (5′-GCGCCCGCGGCGCCATAACAAAGGACACGTCGTACGC-3′), 2.0 μL of 20 μM tRCP reverse—SEQ ID NO: 20 (5′-GCGCCCCGGGCGGTCCGCTAAAAAAAACTGTTTTCTCTTGTTG-3′) and 1.0 μL Expand DNA polymerase. The reactions were overlaid with mineral oil and the thermocycling program was 95° C. for 5 minutes followed by 12 cycles of 95° C. for 30 seconds, 66° C. to 60° C. (minus 0.5° C. every cycle) for 1 minute and 70° C. for 2.5 minutes followed by 25 cycles of 95° C. for 30 seconds, 60° C. for 30 seconds, and 70° C. for 2.5 minutes. The final extension step was 70° C. for 7 minutes. The 500 bp reaction product was gel-purified on 1% TBE agarose, and the DNA was extracted using Qiaprep DNA extraction method. The DNA was cloned into the pCR-BluntII-TOPO vector and completely sequenced.

### Destination Vector

The ZmRCP terminus was cut from the TOPO vector (SacII/Xmae and ligated to a similarly cut pNOV6901 vector, SEQ ID NO: 5. This produced pSYN15861 contains a ZmRCP-GUS assembly. The nucleic acid sequence of pSYN15861 is presented as SEQ ID NO: 7. The complete ZmRCP-GUS expression cassette was mobilized into a binary vector pSYN15605, SEQ ID NO: 6, that had been digested with RsrII followed by treatment with calf alkaline phosphatase as a SanDI/RsrII fragment. This construct, pSYN15888 contains a prZmRCP-GUS-prUBIl-PMI. The nucleic acid sequence of pSYN15888 is presented as SEQ ID NO: 8.

### Example 2

### Construction of Root Cap-Specific Expression Cassettes

### Entry Vectors

The first step in construction of expression cassettes was the cloning of the promoter into an entry vector. PCR primers were designed to amplify the promoter from maize line B73. These isolated nucleotide sequences were TOPO cloned and sequenced. The promoter corresponding to this sequence was designated as Maize Root Cap-Specific 1-2 promoter or ZmRCP1-2 promoter.

This vector is PCR4-Topo containing a putative maize root cap specific promoter prZmRCP1-2. prZmRCP1-2 was PCR-amplified from genomic DNA of maize B73, using primers AG971f—SEQ ID NO: 11 (CTCGAGGGACCCAACAATTTGCCACAAACTGG) and AG972r—SEQ ID NO: 12 (GGATCCTGTAGACTGCTCTGGCTTAA) then cloned into PCR4-Topo and sequenced. The resulting vector is pSYN15670, SEQ ID NO: 21.

### Example 3

### Expression of Gus in Stably-Transformed Corn Directed by Root-Specific Promoters

Maize plants were transformed with an Agrobacterium vector comprising the ZmRCP1 promoter and terminator of the invention operably linked to the GUS coding sequence. The Agrobacterium vector further comprises the Ubiquitin promoter and NOS terminator operably linked to the PMI (Phosphomannose Isomerase) coding sequence.

GUS activity in stably transformed maize was measured by a visual assay. Gus activity was characterized as high (+++), medium (++), low (+), or absent (−) and data from 25 low copy transgenic maize plants were averaged for each promoter construct. Results shown in Table 1 demonstrate that GUS activity in transgenic plants comprising an expression cassette that comprises a promoter of the invention was confined specifically to the roots. The expression is further defined as isolated to the root cap.

TABLE 1
Summary GUS Expression in Tissues Excised
from Transgenic (T0) Maize Plants

GUS Activity in Designated Maize Tissue

| Promoter | Root Cap | Leaf | Silk | Pollen | Kernel |
|---|---|---|---|---|---|
| ZmRCP1 | +++ | − | − | − | − |

### Example 4

### Expression of GUS in T1 Corn Directed by Root-Specific Promoters

The transformed maize plants according to EXAMPLE 2 were grown to flowering and self pollinated. The resulting seed was harvested and dried. Selected T1 seed was germinated The

GUS activity in stably transformed maize was measured by a visual assay. Gus activity was characterized as high (+++), medium (++), low (+), or absent (−) and data from 47 transgenic maize plants were averaged for each promoter construct. Results shown in Table 1 demonstrate that GUS activity in transgenic plants comprising an expression cassette that comprises a promoter of the invention was confined specifically to the roots. The expression is further defined as isolated to the root cap.

TABLE 2
Summary GUS Expression in Tissues Excised
from Transgenic (T1) Maize Plants

GUS Activity in Designated Maize Tissue

| Promoter | Root Cap | Leaf | Silk | Pollen | Kernel |
|---|---|---|---|---|---|
| ZmRCP1 | +++ | − | − | − | − |

TABLE 3
GUS Expression in Root Tissue from Segregating
Transgenic (T1) Maize Plants

| | | Root Tip |
|---|---|---|
| Plant ID | GUS copy No. | Staining |
| 15888-1-1 | 0.0 | NO |
| 15888-1-2 | 1.4 | YES |
| 15888-1-3 | 0.0 | NO |
| 15888-1-4 | 0.0 | NO |
| 15888-1-5 | 0.0 | NO |
| 15888-1-6 | 0.0 | NO |
| 15888-1-7 | 0.9 | YES |
| 15888-1-8 | 1.1 | YES |
| 15888-1-9 | 1.1 | YES |
| 15888-1-10 | 0.0 | NO |
| 15888-1-11 | 1.5 | YES |
| 15888-1-12 | 2.0 | YES |
| 15888-1-13 | 1.3 | YES |
| 15888-1-14 | 1.9 | YES |
| 15888-1-15 | 0.9 | YES |
| 15888-1-16 | 0.9 | YES |
| 15888-1-17 | 0.0 | NO |
| 15888-1-18 | 0.0 | NO |
| 15888-1-19 | 0.0 | NO |
| 15888-1-20 | 1.9 | YES |
| 15888-1-21 | 1.0 | YES |
| 15888-1-22 | 1.1 | YES |
| 15888-2-1 | 0.9 | YES |
| 15888-2-2 | 0.0 | NO |
| 15888-2-3 | 0.0 | NO |
| 15888-2-4 | 2.3 | YES |
| 15888-2-5 | 0.0 | NO |
| 15888-2-6 | 1.2 | YES |
| 15888-2-7 | 1.8 | YES |
| 15888-2-8 | 1.0 | YES |
| 15888-2-9 | 1.0 | YES |
| 15888-2-10 | 1.0 | YES |
| 15888-3-1 | 0.9 | YES |
| 15888-3-2 | 0.0 | NO |
| 15888-3-3 | 0.9 | YES |
| 15888-3-4 | 0.8 | YES |
| 15888-3-5 | 2.1 | YES |
| 15888-3-6 | 1.8 | YES |
| 15888-3-7 | 2.2 | YES |
| 15888-3-8 | 1.1 | YES |
| 15888-3-9 | 1.6 | YES |
| 15888-3-10 | 0.0 | NO |
| 15888-3-11 | 0.0 | NO |
| 15888-3-12 | 0.0 | NO |
| 15888-3-13 | 0.4 | YES |
| 15888-3-15 | 1.7 | YES |
| 15888-3-16 | 0.0 | NO |

What is claimed is:

1. An isolated nucleic acid molecule capable of directing expression in plant cell, wherein said nucleic acid molecule comprises a promoter set forth in SEQ ID NO: 1

2. The isolated nucleic acid molecule according to claim 1, wherein said promoter is capable of driving root cap-specific expression of an operably linked nucleotide sequence.

3. The isolated nucleic acid molecule according to claim 1, wherein said nucleic acid molecule is isolated from the root tissue of a target plant species.

4. The isolated nucleic acid molecule according to claim 3, wherein said target plant species is maize.

5. An expression cassette comprising, in sequence, the nucleic acid molecule of claim 1 operably linked to a heterologous coding sequence, which is operably linked to a 3′-untranslated region including a polyadenylation signal.

6. The expression cassette according to claim 5, wherein said heterologous coding sequence is selected from the group consisting of insecticidal coding sequences, nematicidal coding sequences, herbicide tolerance coding sequences, anti-microbial coding sequences, anti-fungal coding sequences, anti-viral coding sequences, abiotic stress tolerance coding sequences, nutritional quality coding sequences, visible marker coding sequences and selectable marker coding sequences.

7. The expression cassette according to claim 6, wherein said insecticidal coding sequence encodes a toxin active against a coleopteran pest.

8. The expression cassette according to claim 7, wherein said coleopteran pest is a species in the genus Diabrotica.

9. The expression cassette according to claim 6, wherein said visible marker is β-glucuronidase.

10. A recombinant vector comprising the expression cassette of claim 6.

11. The recombinant vector according to claim 10, wherein said vector is a plasmid.

12. A transgenic non-human host cell comprising the expression cassette of claim 6.

13. The transgenic non-human host cell according to claim 12, which is a transgenic plant cell.

14. A transgenic plant comprising the transgenic plant cell of claim 13.

15. The transgenic plant according to claim 14, wherein said plant is selected from the group consisting of sorghum, wheat, sunflower, tomato, cole crops, cotton, rice, soybean, sugar beet, sugarcane, tobacco, barley, oilseed rape and maize.

16. The transgenic plant according to claim 15, wherein said plant is a maize plant.

17. The transgenic plant according to claim 15, wherein said plant is a rice plant.

18. Transgenic seed from the transgenic plant of claim 15.

19. Transgenic seed from the maize plant of claim 16.

20. Transgenic seed from the rice plant of claim 17.

21. A method of specifically expressing a heterologous coding sequence in transgenic plant roots comprising:
(a) transforming plant cells with a vector, wherein said vector comprises the expression cassette of claim 5,
(b) growing the transgenic plant cells comprising said expression cassette, and
(c) producing transgenic plants from said transformed plant cells wherein the heterologous coding sequence is specifically expressed in plant roots under control of said nucleic acid molecule.

22. The method according to claim 21, wherein the transgenic plant roots are maize or rice roots.

23. The method according to claim 21, wherein said heterologous coding sequence is selected from the group consisting of insecticidal coding sequences, nematicidal coding sequences, herbicide tolerance coding sequences, anti-microbial coding sequences, anti-fungal coding sequences, anti-viral coding sequences, abiotic stress tolerance coding sequences, nutritional quality coding sequences, visible marker coding sequences and selectable marker coding sequences.

24. A transgenic plant produced according to the method of claim 21.

25. The transgenic plant according to claim 24, wherein said plant is a monocot.

26. The plant of claim 25, wherein said monocot is maize.

27. The plant of claim 25, wherein said monocot is rice.


SEQUENCE LISTING (21 sequences)

SEQ ID NO: 1 - DNA, 1537 bp, Zea mays
aacaatttgccacaaactggcaattagattttcttgtcgtctgctggcaacacgacatcactttcggcgtcacttagcaattttttctagtgatgataaggtctagcaggtgtttgatatggctccaaaataaaactccaaagaagagatgatcaaagctagccaaacaacctaactctccaaggaatttcaggaattgcaatataatttttttgtagtgcatctttgctgctccgaaatctagaaaactgtagagctactccatgctaaagctgtagagccgagccatcttcaattctgataaaaaagcccaagactagctagctctctagcacaagacaagacaaaaaaaaaacacgccttctttcctttcgagctagtaaacttttttcccacaagaaatggtcattactgaacccccttttgtgattcattatattggaatacccaatactagaagggaaaaaaatttaaaggtggtcacattccacatccaaagataaagtgaatattaattgtcatgtatgatcctttatcaatttgcctcgatctccatagcgaaacaactcactttttttggtaagtgaaacttgtttgttgttattaattttcaacataaaaaaaactcacttttttttatattctttgcttgcttcgaattatatcaattgtacgtggtcgatactcgatagtatgatgcatgctatccggctaactaacatgcatgcatgggatggggatgggagagcaagttggggttggacgctttccttgctgctcaaggagcagctagctagcagcttgctatagaaagtaggcattgcttttaatgcaatcgcacatcatcgatctaatattaatcttggccgtggcgttgaccttcttgcatgatatggttggtccggcgatggatggacatgcatgcatgcaacagctagtacgtacgacgtactacgtgcaggttgcttacaaacaagagatctgaatggaagagcatgcatttgctgatccgggactcaagggccaatccatccatccccagccggccgcgatgtgtaaatgtcaactgaagatgcaggctagggacatgcatgcattatatatataagctacctccagctttggctgcgatcaagacaccaattaagccagagcagtctacaatggctcacagtcacagaagcagtatgagcttctgcttggtgtgcatcgtcgtcatggcggcggcggcggcgttgttgcaacccgcatcttgacagcaggcggtgccggacatcccgaacctgtaggtgggctccaacgatcgcagcacgactctctagtgcaccaacatcaagagcaacaagaccacctgcagcgccacctgcaacgcacgctgccctcacaagtgcctcatccagtgcccaagctgcaagacattctgctgtacgtacggatcggagtatttcatttcgtccatcttaacaattaatttggttcctttccatcttaataatttgatatatatgcacatgcagtgtgcgacttgaatcccggc

SEQ ID NO: 2 - DNA, 1163 bp, Zea mays
aacaatttgccacaaactggcaattagattttcttgtcgtctgctggcaacacgacatcactttcggcgtcacttagcaattttttctagtgatgataaggtctagcaggtgtttgatatggctccaaaataaaactccaaagaagagatgatcaaagctagccaaacaacctaactctccaaggaatttcaggaattgcaatataatttttttgtagtgcatctttgctgctccgaaatctagaaaactgtagagctactccatgctaaagctgtagagccgagccatcttcaattctgataaaaaagcccaagactagctagctctctagcacaagacaagacaaaaaaaaaacacgccttctttcctttcgagctagtaaacttttttcccacaagaaatggtcattactgaacccccttttgtgattcattatattggaatacccaatactagaagggaaaaaaatttaaaggtggtcacattccacatccaaagataaagtgaatattaattgtcatgtatgatcctttatcaatttgcctcgatctccatagcgaaacaactcactttttttggtaagtgaaacttgtttgttgttattaattttcaacataaaaaaaactcacttttttttatattctttgcttgcttcgaattatatcaattgtacgtggtcgatactcgatagtatgatgcatgctatccggctaactaacatgcatgcatgggatggggatgggagagcaagttggggttggacgctttccttgctgctcaaggagcagctagctagcagcttgctatagaaagtaggcattgcttttaatgcaatcgcacatcatcgatctaatattaatcttggccgtggcgttgaccttcttgcatgatatggttggtccggcgatggatggacatgcatgcatgcaacagctagtacgtacgacgtactacgtgcaggttgcttacaaacaagagatctgaatggaagagcatgcatttgctgatccgggactcaagggccaatccatccatccccagccggccgcgatgtgtaaatgtcaactgaagatgcaggctagggacatgcatgcattatatatataagctacctccagctttggctgcgatcaagacaccaattaagccagagcagtctaca

SEQ ID NO: 3 - DNA, 1163 bp, Zea mays; identical to SEQ ID NO: 2

SEQ ID NO: 4 - DNA, 1225 bp, Zea mays
tctgcgatcaagacaccaattaagccagagcagtctacaatggctcacagtcacagaagcagtatgagcttctgcttggtgtgcatcgtcgtcatggcggcggcggcggcgttgttgcaacccgcatcttctcagcaggcggtgccggacatcccgaacctgaaggtgggctccaacgatcgcagcacgactctcaagtgcaccaacatcaagagcaacaagaccacctgcagcgccacctgcaacgcacgctgccctcacaagtgcctcatccagtgcccaagctgcaagacattctgcttgtgcgacttctatcccggcgtgtcctgcggcgacccacgcttcacgggcgccgacggcaacaacttctacttccacggcaagaaggaccgggacttctgcatcgtctccgacgccgccctccacatcaacgcgcacttcatcggcaagcgcaacccggccatgagccgcgacttcacctggatccaggcgctgggcatccgcttcgcgcaccaccacctctacgtcgccgcccagaggacgcccaggtgggacgccgccgccgaccacctggcgctggccctcgacgacgaggacgtcgacgtcgcgtccctgctgccgcgcttcgtcggcgcgcgctggtccccgcccacggcgccggcgctgtccgtcacccgcaccgcgcgcgtcaacaccgtcgtcgtcgagctcaggggcgccttccgcatcgtcgccagcgtagtgcccatcaccgccgaggactcgcggatccacaactacggcgtcagggaggacgacggcgacaccctcgcgcacctcgacctcggcttcaagttctacgacctcaccgacgacgtgcacggcgtgctgggccagacctaccgcccggactacgtcaacagcctcaacgtcacatccaacatgcccgtcatgggaggcgcgccggactacctctcctccgacctcttctccaccgactgcgccgtcgcacgcttcggcggacgccgacaccagcaagccaccgccgctaatattgccatgctcaccgacgacgacgacatggaatgaataacaaaggacacgtcgtacgcgcgatcgacgactttaccgtaccatttcattaccatctccgaggatatcgatccatcgttgtgtgtcttctacactagctagtgaatgaagaagcagcaataaacccattgaaattatatattatatattcaac

SEQ ID NO: 5 - DNA, 4700 bp, Artificial
  pNOV6901 Vector
tggcttatcgaaattaatacgactcactatagggagaccggcctcgagcagctgaagcttgcatgcctgcaggtcgactctagaggatccccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctccgcgggcggccgcactagtcccgggcccatcgatgatatcagatctggttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaacc

SEQ ID NO: 6 - DNA, 9522 bp, Artificial
  pSYN15605 Vector sequence
aattcctgtggttggcatgcacatacaaatggacgaacggataaaccttttcacgcccttttaaatatccgattattctaataaacgctcttttctcttaggtttacccgccaatatatcctgtcaaacactgatagtttaaacgggacccggcgcgccatttaaatggtaccggacccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgccggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatt

SEQ ID NO: 7 - DNA, 6710 bp, Artificial
  pSYN15861 Vector sequence
tcgagggacccaacaatttgccacaaactggcaattagattttcttgtcgtctgctggcaacacgacatcactttcggcgtcacttagcaattttttctagtgatgataaggtctagcaggtgtttgatatggctccaaaataaaactccaaagaagagatgatcaaagctagccaaacaacctaactctccaaggaatttcaggaattgcaatataatttttttgtagtgcatctttgctgctccgaaatctagaaaactgtagagctactccatgctaaagctgtagagccgagccatcttcaattctgataaaaaagcccaagactagctagctctctagcacaagacaagacaaaaaaaaaacacgccttctttcctttcgagctagtaaacttttttcccacaagaaatggtcattactgaacccccttttgtgattcattatattggaatacccaatactagaagggaaaaaaatttaaaggtggtcacattccacatccaaagataaagtgaatattaattgtcatgtatgatcctttatcaatttgcctcgatctccatagcgaaacaactcactttttttggtaagtgaaacttgtttgttgttattaattttcaacataaaaaaaactcacttttttttatattctttgcttgcttcgaattatatcaattgtacgtggtcgatactcgatagtatgatgcatgctatccggctaactaacatgcatgcatgggatggggatgggagagcaagttggggttggacgctttccttgctgctcaaggagcagctagctagcagcttgctatagaaagtaggcattgcttttaatgcaatcgcacatcatcgatctaatattaatcttggccgtggcgttgaccttcttgcatgatatggttggtccggcgatggatggacatgcatgcatgcaacagctagtacgtacgacgtactacgtgcaggttgcttacaaacaagagatctgaatggaagagcatgcatttgctgatccgggactcaagggccaatccatccatccccagccggccgcgatgtgtaaatgtcaactgaagatgcaggctagggacatgcatgcattatatatataagctacctccagctttggctgcgatcaagacaccaattaagccagagcagtctacaatggctcacagtcacagaagcagtatgagcttctgcttggtgtgcatcgtcgtcatggcggcggcggcggcgttgttgcaacccgcatcttgacagcaggcggtgccggacatcccgaacctgtaggtgggctccaacgatcgcagcacgactctctagtgcaccaacatcaagagcaacaagaccacctgcagcgccacctgcaacgcacgctgccctcacaagtgcctcatccagtgcccaagctgcaagacattctgctgtacgtacggatcggagtatttcatttcgtccatcttaacaattaatttggttcctttccatcttaataatttgatatatatgcacatgcagtgtgcgacttgaatcccggcggcgccggatccccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctccgcggcgccataacaaaggacacgtcgtacgcgcgatcgacgacttcaccgtaccattccattaccatctcggaggatatcgatccatccatcgttgtgtgtcttctacactagctagtgaatgaagaagcagcaataaacccattgaaattatatattatatattcaacaaataaggcttataaatacatatgcatgcatgtatactcctaattaattaattaaatactcctgattttattttacaatgcatggatgtttaagtaatcgatcgagcttcacttgatagttcgtggaattaagaaactgagcacaagataaaatgtaatcgagtgcttaacaattttgggactctcatcatcacaactcacaagcaaaacaaaaatggaaacaaacaattttagtttcttggtgggccttggcagtgtggagttgggcctgtggctcgtctaactcgatccggaggcccatctcacgaagtcacaacaagagaaaacagttttttttagcggaccgcccgggcccatcgatgatatcagatctggttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaacctggcttatcgaaattaatacgactcactatagggagaccggcc

SEQ ID NO: 8 - DNA, 13816 bp, Artificial
  pSYN15888 Vector sequence
attcctgtggttggcatgcacatacaaatggacgaacggataaaccttttcacgcccttttaaatatccgattattctaataaacgctcttttctcttaggtttacccgccaatatatcctgtcaaacactgatagtttaaacgggacccggcgcgccatttaaatggtaccggacccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgccggacccaacaatttgccacaaactggcaattagattttcttgtcgtctgctggcaacacgacatcactttcggcgtcacttagcaattttttctagtgatgataaggtctagcaggtgtttgatatggctccaaaataaaactccaaagaagagatgatcaaagctagccaaacaacctaactctccaaggaatttcaggaattgcaatataatttttttgtagtgcatctttgctgctccgaaatctagaaaactgtagagctactccatgctaaagctgtagagccgagccatcttcaattctgataaaaaagcccaagactagctagctctctagcacaagacaagacaaaaaaaaaacacgccttctttcctttcgagctagtaaacttttttcccacaagaaatggtcattactgaacccccttttgtgattcattatattggaatacccaatactagaagggaaaaaaatttaaaggtggtcacattccacatccaaagataaagtgaatattaattgtcatgtatgatcctttatcaatttgcctcgatctccatagcgaaacaactcactttttttggtaagtgaaacttgtttgttgttattaattttcaacataaaaaaaactcacttttttttatattctttgcttgcttcgaattatatcaattgtacgtggtcgatactcgatagtatgatgcatgctatccggctaactaacatgcatgcatgggatggggatgggagagcaagttggggttggacgctttccttgctgctcaaggagcagctagctagcagcttgctatagaaagtaggcattgcttttaatgcaatcgcacatcatcgatctaatattaatcttggccgtggcgttgaccttcttgcatgatatggttggtccggcgatggatggacatgcatgcatgcaacagctagtacgtacgacgtactacgtgcaggttgcttacaaacaagagatctgaatggaagagcatgcatttgctgatccgggactcaagggccaatccatccatccccagccggccgcgatgtgtaaatgtcaactgaagatgcaggctagggacatgcatgcattatatatataagctacctccagctttggctgcgatcaagacaccaattaagccagagcagtctacaatggctcacagtcacagaagcagtatgagcttctgcttggtgtgcatcgtcgtcatggcggcggcggcggcgttgttgcaacccgcatcttgacagcaggcggtgccggacatcccgaacctgtaggtgggctccaacgatcgcagcacgactctctagtgcaccaacatcaagagcaacaagaccacctgcagcgccacctgcaacgcacgctgccctcacaagtgcctcatccagtgcccaagctgcaagacattctgctgtacgtacggatcggagtatttcatttcgtccatcttaacaattaatttggttcctttccatcttaataatttgatatatatgcacatgcagtgtgcgacttgaatcccggcggcgccggatccccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctccgcggcgccataacaaaggacacgtcgtacgcgcgatcgacgacttcaccgtaccattccattaccatctcggaggatatcgatccatccatcgttgtgtgtcttctacactagctagtgaatgaagaagcagcaataaacccattgaaattatatattatatattcaacaaataaggcttataaatacatatgcatgcatgtatactcctaattaattaattaaatactcctgattttattttacaatgcatggatgtttaagtaatcgatcgagcttcacttgatagttcgtggaattaagaaactgagcacaagataaaatgtaatcgagtgcttaacaattttgggactctcatcatcacaactcacaagcaaaacaaaaatggaaacaaacaattttagtttcttggtgggccttggcagtgtggagttgggcctgtggctcgtctaactcgatccggaggcccatctcacgaagtcacaacaagagaaaacagttttttttagcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 9 - DNA, 38 bp, Artificial
  Primer sequence
gctagcctcgagggacccaacaatttgccacaaactgg

SEQ ID NO: 10 - DNA, 39 bp, Artificial
  Primer sequence
gctagcggatccggcgccgccgggatagaagtcgcacac

SEQ ID NO: 11 - DNA, 32 bp, Artificial
  Primer sequence
ctcgagggacccaacaatttgccacaaactgg

SEQ ID NO: 12 - DNA, 26 bp, Artificial
  Primer sequence
ggatcctgtagactgctctggcttaa

SEQ ID NO: 13 - DNA, 29 bp, Artificial
  Primer sequence
gcggcggcggcgtagttgcaacccgcatc

SEQ ID NO: 14 - DNA, 30 bp, Artificial
  Primer sequence
gcagtgtgcgacttgaatcccggcggcgcc

SEQ ID NO: 15 - DNA, 29 bp, Artificial
  Primer sequence
ctactccatgctaaagctgtagagccgag

SEQ ID NO: 16 - DNA, 29 bp, Artificial
  Primer sequence
cctttatcaatttgcctcgatctccatag

SEQ ID NO: 17 - DNA, 30 bp, Artificial
  Primer sequence
gaaacttgtttgttgttattaattttcaac

SEQ ID NO: 18 - DNA, 29 bp, Artificial
  Primer sequence
gcaccaacatcaagagcaacaagaccacc

SEQ ID NO: 19 - DNA, 37 bp, Artificial
  Primer sequence
gcgcccgcggcgccataacaaaggacacgtcgtacgc

SEQ ID NO: 20 - DNA, 43 bp, Artificial
  Primer sequence
gcgccccgggcggtccgctaaaaaaaactgttttctcttgttg

SEQ ID NO: 21 - DNA, 5137 bp, Artificial
  pSYN15670 vector
actaaagggactagtcctgcaggtttaaacgaattcgcccttaagggcggatcctgtagactgctctggcttaattggtgtcttgatcgcagccaaagctggaggtagcttatatatataatgcatgcatgtccctagcctgcatcttcagttgacatttacacatcgcggccggctggggatggatggattggcccttgagtcccggatcagcaaatgcatgctcttccattcagatctcttgtttgtaagcaacctgcacgtagtacgtcgtacgtactagctgttgcatgcatgcatgtccatccatcgccggaccaaccatatcatgcaagaaggtcaacgccacggccaagattaatattagatcgatgatgtgcgattgcattaaaagcaatgcctactttctatagcaagctgctagctagctgctccttgagcagcaaggaaagcgtccaaccccaacttgctctcccatccccatcccatgcatgcatgttagttagccggatagcatgcatcatactatcgagtatcgaccacgtacaattgatataattcgaagcaagcaaagaatataaaaaaaagtgagttttttttatgttgaaaattaataacaacaaacaagtttcacttaccaaaaaaagtgagttgtttcgctatggagatcgaggcaaattgataaaggatcatacatgacaattaatattcactttatctttggatgtggaatgtgaccacctttaaatttttttcccttctagtattgggtattccaatataatgaatcacaaaagggggttcagtaatgaccatttcttgtgggaaaaaagtttactagctcgaaaggaaagaaggcgtgttttttttttgtcttgtcttgtgctagagagctagctagtcttgggcttttttatcagaattgaagatggctcggctctacagctttagcatggagtagctctacagttttctagatttcggagcagcaaagatgcactacaaaaaaattatattgcaattcctgaaattccttggagagttaggttgtttggctagctttgatcatctcttctttggagttttattttggagccatatcaaacacctgctagaccttatcatcactagaaaaaattgctaagtgacgccgaaagtgatgtcgtgttgccagcagacgacaagaaaatctaattgccagtttgtggcaaattgttgggtccctcgaggaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctc